(19) United States
(12) Patent Application Publication
(10) Pub. No.: US 2018/0036173 A1
(43) Pub. Date: Feb. 8, 2018

(54) OCULAR FILTRATION DEVICES, SYSTEMS AND METHODS

(71) Applicant: The Regents of the University of Colorado, a body corporate, Denver, CO (US)

(72) Inventors: Jeffrey Olson, Denver, CO (US); Ramanath Bhandari, Springfield, IL (US)

(73) Assignee: The Regents of the University of Colorado, a body corporate, Denver, CO (US)

(21) Appl. No.: 15/785,299

(22) Filed: Oct. 16, 2017

Related U.S. Application Data
(63) Continuation of application No. PCT/US2016/027880, filed on Apr. 15, 2016.
Application No. PCT/US2016/027880 is a continuation-in-part of application No. 14/435,407, filed on Apr. 13, 2015.
(60) Provisional application No. 62/148,594, filed on Apr. 16, 2015.
(60) Provisional application No. 61/769,443, filed on Feb. 26, 2013.
(60) Provisional application No. 61/712,511, filed on Oct. 11, 2012.

(51) Int. Cl. A61F 9/007

(52) U.S. Cl. CPC A61F 9/00781

(57) ABSTRACT

A glaucoma drainage device regulator (GDDR) is disclosed which comprises a membrane and a shunt tube to regulate the flow of aqueous in conjunction with different ocular (e.g., glaucoma) filtering procedures. In connection with aqueous shunting, the membrane of the shunt tube of the GDDR can be placed in the anterior chamber of the eye during implantation and coupled to a reservoir. The GDDR is implanted in a manner to permit easy access to the membrane for post surgery perforation of the membrane to regulate the aqueous flow of the shunt tube.

### CROSS REFERENCE TO RELATED APPLICATIONS

This U.S. Non-Provisional patent application is a continuation of and claims priority to International Application No. PCT/US2016/027880, filed on Apr. 15, 2016, entitled “OCULAR FILTRATION DEVICES, SYSTEMS AND METHODS,” which claims priority to U.S. Provisional Application No. 62/148,594, filed on Apr. 16, 2015, entitled “OCULAR FILTRATION DEVICES, SYSTEMS AND METHODS.” This U.S. Non-Provisional patent application is also a continuation-in-part of and claims priority to U.S. Non-Provisional patent application Ser. No. 14/435,407, filed on Apr. 13, 2015, entitled “OCULAR FILTRATION DEVICES, SYSTEMS AND METHODS,” which is a U.S. National Stage Entry under 35 U.S.C. §371 of International Application No. PCT/US2013/64473, filed on Oct. 11, 2013, entitled “OCULAR FILTRATION DEVICES, SYSTEMS AND METHODS,” which claims priority to both U.S. Provisional Application No. 61/769,443, filed on Feb. 26, 2013, entitled “OCULAR FILTRATION DEVICES, SYSTEMS AND METHODS,” and U.S. Provisional Application No. 61/712,511, filed on Oct. 11, 2012, entitled “OCULAR FILTRATION DEVICES, SYSTEMS AND METHODS.” All of the foregoing are incorporated herein by reference in their entireties.

### FIELD

The present disclosure relates to ocular filtration devices, systems and methods, and more particularly, to glaucoma treatment devices, systems and methods.

### DISCUSSION OF THE RELATED ART

Glaucoma is a rapidly growing problem in the industrialized world and presents a leading cause of vision loss and blindness. Currently, glaucoma is the second leading cause of irreversible blindness. Glaucoma prevalence is currently approximately 2.2 million people in the United States and over 60 million worldwide. Despite recent technological and pharmacologic advances in medicine, the number of people losing sight due to glaucoma continues to increase.

In brief, glaucoma is characterized by high intraocular pressures, which over time cause damage to the optic nerve, resulting in loss of peripheral vision in early cases. Later stage disease can lead to loss of central vision and permanent blindness. Treatment is aimed at lowering intraocular pressure.

The current standard of care for treating the blinding complications of glaucoma revolves around topical medications, laser treatments, and surgery for the most advanced cases, all aimed at lowering intraocular pressure. For patients with advanced disease, filtering surgery (e.g., aqueous shunting or trabeculectomy) is often required to prevent vision loss.

With respect to aqueous shunting, implanted glaucoma drainage devices (GDDs) are typically used to create an alternate aqueous pathway from the anterior chamber by shunting aqueous out of the eye through a tube to a subconjunctival bleb or reservoir which is usually connected to a plate under the conjunctiva. A major disadvantage of this surgery is that the aqueous may tend to flow too rapidly out of the tube until a fibrous membrane has encapsulated the reservoir. To this end, medical practitioners may elect to tie off the external portion of the tube or block its lumen with suture or other material, such that once the reservoir has become encapsulated, the suture can be removed. These represent an all-or nothing option with regards to the amount of aqueous flow. Further, some GDDs have a valve which theoretically prevents flow below certain pressures, but cannot be titrated or adjusted by the medical practitioner.

As with conventional GDD implantation, current trabeculectomy surgeries are not titratable by the medical practitioner post-operatively. During surgery, viscoelastic substances may be left in the anterior chamber to slow the rate of aqueous filtration for the first 24-48 hours, or contact lenses placed on the surface of the eye post-operatively to prevent low pressures. Alternatively, the medical practitioner may place sutures over the sclerostomy flap, and can open these with a laser or mechanically. Again, these allow the medical practitioner to either prevent or allow flow, but without precision, often leading to gross under- or over-filtration. This problem contributes to the high rate of surgical failure with these surgeries long-term.

At least in part due to not being titratable, current surgical techniques are plagued by high rates of complications (such as overfiltering and underfiltering, hypotony, choroidal effusions/hemorrhages), with a failure rate of 50% at 5 years. To address this issue, there exist prior art of using biodegradable implants, fibroblast inhibitors, anti-metabolites, and other drugs over the surface of the scleral flap or stainless steel shunts under the scleral flap to encourage continued flow. For example, the Ex-Press Mini Glaucoma Shunt was originally developed by Optonol, Ltd. (Neve Ilan, Israel) for implantation under the conjunctiva for controlling intraocular pressure (TOP). This biocompatible device is almost 3 mm long with an external diameter of approximately 400 microns. It is a non-valved, MRI compatible, stainless steel device with a 50 micron lumen. It has an external disc at one end and a spur-like extension on the other to prevent extrusion.

### SUMMARY

A glaucoma drainage device regulator (GDDR) is disclosed which comprises a membrane and a lumen to regulate the flow of aqueous in conjunction with different ocular (e.g., glaucoma) filtering procedures. In connection with aqueous shunting, the GDDR can be placed over the tip of a shunt tube in the anterior chamber, either at the time of initial surgery or also in devices which have been previously implanted. In connection with trabeculectomy, the GDDR can comprise a flange for seating the GDDR at the sclerostomy in trabeculectomy surgery.

### BRIEF DESCRIPTION OF THE DRAWINGS

The accompanying drawings are included to provide a further understanding of the disclosure and are incorporated in and constitute a part of this specification, illustrate embodiments of the disclosure, and together with the description serve to explain the principles of the disclosure, in which like numerals denote like elements and:

FIG. 1 illustrates views of a GDDR in accordance with the present disclosure;

FIG. 2 illustrates exploded and coupled views of a GDDR, a shunt tube, and a reservoir in accordance with the present disclosure;

FIG. 3A illustrates a GDDR system in accordance with the present disclosure implanted in connection with aqueous shunting;

FIG. 3B illustrates another GDDR system in accordance with the present disclosure implanted in connection with aqueous shunting and a multi-lumen or bifurcated shunt tube;

FIG. 4 illustrates a GDDR comprising a flange in accordance with the present disclosure;

FIG. 5 illustrates progressive views of a GDDR comprising a flange implanted in connection with trabeculectomy in accordance with the present disclosure;

FIG. 6 illustrates a GDDR in accordance with the present disclosure implanted in connection with trabeculectomy;

FIG. 7 illustrates in vitro test results;

FIG. 8 illustrates ex-vivo test results;

FIG. 9 illustrates another GDDR system having an integral shunt tube with a closed distal end and a winged proximal end with one or more protuberances running along the length of the tube in accordance with the present disclosure;

FIG. 10 illustrates a side perspective view of the GDDR system of FIG. 9, showing the winged proximal end of the shunt tube;

FIG. 11 illustrates a proximal end view of the winged shunt tube mated with the reservoir of the GDDR system of FIG. 9;

FIG. 12 illustrates a closed distal end view of the shunt tube of the GDDR system of FIG. 9; and

FIG. 13 illustrates another view of the GDDR system of FIG. 9.

FIG. 14 illustrates flow through a large lumen glaucoma drainage device (LL-GDD) increases exponentially as the membrane cap is opened with laser. For comparison, the flow of a standard glaucoma drainage device is depicted by the horizontal bar.

FIG. 15 illustrates drop on TOP after the initial surgical implantation the first membrane lasering, and the second membrane lasering, demonstrating an ability to lower the IOP non-invasively on-demand.

### DETAILED DESCRIPTION OF THE ILLUSTRATED EMBODIMENTS

Persons skilled in the art will readily appreciate that various aspects of the present disclosure can be realized by any number of methods and systems configured to perform the intended functions. Stated differently, other methods and systems can be incorporated herein to perform the intended functions. It should also be noted that the accompanying drawing figures referred to herein are not all drawn to scale, but may be exaggerated to illustrate various aspects of the present disclosure, and in that regard, the drawing figures should not be construed as limiting. Finally, although the present disclosure can be described in connection with various principles and beliefs, the present disclosure should not be bound by theory.

As noted above, the flow rate of prior art devices cannot be accurately controlled or adjusted once implanted to fit the needs of the patient. What is therefore needed is a device which could allow the medical practitioner to precisely control the filtration flow rate at some later time, months to years after surgery, decreasing surgical complications, the need for further surgeries, and improving patient outcomes.

The present disclosure obviates these drawbacks and others by allowing medical practitioners to post-operatively control the rate of flow through the device, allowing better, customized treatment for patients. The rate of flow through a tube can be expressed by Poiseuille's law, which states that flow is proportional to the radius raised to the fourth power. Consequently, small changes in the radius of the tube produce large changes in flow.

In an example embodiment, a glaucoma drainage device regulator (GDDR) comprises a membrane connected a lumen. In an example embodiment, the GDDR further comprises a flange. The GDDR is configured to be implanted in an eye to regulate aqueous flow from the anterior chamber and/or to lower intraocular pressure. In an example embodiment, the membrane is configured with perforations. In another example embodiment, the membrane is configured to be perforated post implantation, perhaps long after implantation. The perforations are configured, in an example embodiment, to increase the flow of aqueous from the anterior chamber and/or to lower intraocular pressure in a controllably adjustable manner. The lumen can, in an example embodiment, be coupled to the end of a shunt tube and/or reservoir.

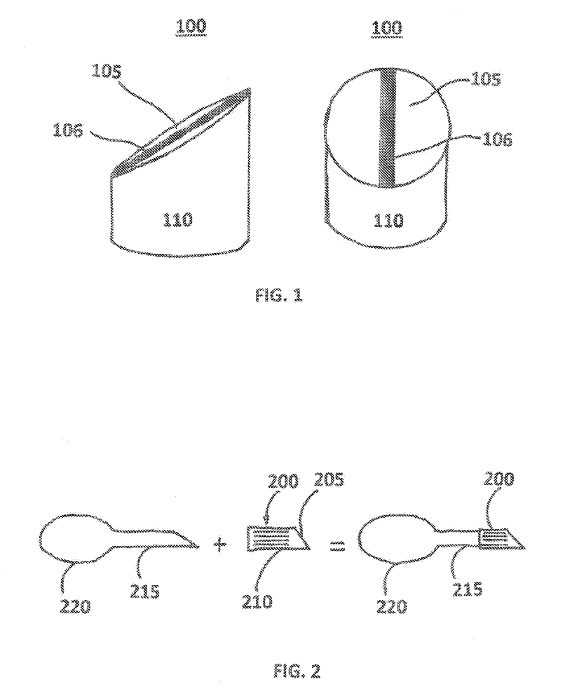

With reference to FIG. 1, a GDDR 100 is disclosed which uses a membrane 105 to regulate the flow of aqueous in conjunction with different ocular (e.g., glaucoma) filtering procedures. Membrane 105 of GDDR 100 can be comprised of one or more biocompatible materials such as PVDF, silicone, filtration and nanofiltration membranes, nucleopore membranes, PMMA, dialysis membranes, cellulose, acrylic, fluorinated ethylene propylene, shape memory polymers, non-reactive polymers, collamers, nylon, and the like. Membrane 105 may be biocompatible plant or animal living cells. Membrane 105 may be grown of living cells on a scaffold, molded, made using 3-D printing, or other known manufacturing means. Membrane 105 can be configured such that it allows no aqueous flow prior to perforation, or it may be permeable to low amounts of aqueous flow prior to perforation.

In example embodiments, a surface of membrane 105 can be color coded, numbered, or have writing or another target to indicate one or more areas to perforate in order to achieve a certain amount of flow, or to access different drainage areas, tubes, and/or shunts.

In some embodiments, per the medical practitioner's discretion, acoustic (e.g., ultrasound), thermal, photodisruptive or ablastive laser (Nd:Yag, argon, PASCAL, etc.) can be used either directly or with the use of a mirrored lens or other optically coupled focusing mechanism to pass through overlying tissue and create small perforations or ruptures in the surface of membrane 105, thereby allowing the passage of aqueous. In other embodiments, membrane 105 can be perforated mechanically such as with a needle or other sharp instrument.

In another example embodiment, membrane 105 can be configured to dissolve or be dissolved to facilitate increased passage of aqueous. In one example embodiment, membrane 105 can partially dissolve to increase the flow of aqueous or reduce intraocular pressure. In another example, specific portions of membrane 105 may fully dissolve to increase the flow of aqueous or reduce intraocular pressure. Biodegradable or bioabsorbing materials, such as collagen can be used to this end.

A perforation can comprise a hole, a slit, or any physical change to membrane 105 that facilitates increased aqueous flow through membrane 105 and/or lowering intraocular pressure. In an example embodiment, any suitable number of perforations can be made in membrane 105. In an example embodiment, the perforations can be any suitable size or shape. Perforations can be created in any number of patterns to regulate the flow of aqueous. In an example embodiment, membrane 105 is configured to be perforated by the medical practitioner so that an increase in the number of perforations facilitates an increase in the rate of flow, allowing a titration of aqueous flow based on the clinical need.

In an example embodiment, membrane 105 comprises dividers 106. Dividers 106 are configured to allow the medical practitioner to perforate specific areas selectively (e.g., dividers 106 that correspond to a plurality of lumens, or multi-lumen or bifurcated lumens in connection with aqueous shunting). In another example embodiment, membrane 105 may comprise a continuous face. In this example embodiment, the medical practitioner can still perforate specific areas selectively to further reduce intraocular pressure, as desired. Membrane 105 can be configured as a cap to one or more lumens in connection with aqueous shunting.

Membrane 105 can be impregnated with medicants, such as steroids or others that inhibit fibroblast proliferation, or anti-glaucoma medicants, that are released upon perforation or slow time release. In the alternative, or in addition, these same medicants can be sequestered behind membrane 105 and be configured to be released upon perforation.

With continued reference to FIG. 1, GDDR 100 further comprises a lumen 110. Lumen 110 of GDDR 100 can be comprised of one or more biocompatible materials such as silicone, acrylic, PMMA, fluorinated ethylene propylene, stainless surgical steel, shape memory polymers, collamers, PVDF, bioidentical plant or animal cells, and the like.

In various embodiments, membrane 105 is angled relative to lumen 110. For example, membrane 105 can be configured to be angled relative to the longitudinal axis of lumen 110 at about 30 to about 60 degrees, or at about 45 degrees. Moreover, membrane 105 can be configured to be angled relative to the longitudinal axis of lumen 110 at any suitable angle, including a perpendicular configuration at 0 degrees. In one example embodiment, the angle is selected to increase the surface area of membrane 105. In another example embodiment, the angle is selected to facilitate perforating membrane 105. The angle can allow the surgeon easier surgical access to the face of membrane 105 in order to use a laser or other device to create perforations.

Turning now to FIG. 2, in connection with various embodiments, a lumen 210 of a GDDR 200 can be placed over the tip of a shunt tube 215 in the anterior chamber, either at the time of initial surgery or also in devices which have been previously implanted. In this regard, lumen 210 can be generally configured to sealingly couple with one or more shunt tubes 215. In example embodiments, one or more shunt tubes 215 can be part of conventional glaucoma drainage devices so as to retrofit or be an accessory to the same.

In other example embodiments, lumen 210 can be placed over “minimally-invasive glaucoma devices” or MIGS, for example, a micro-bypass stent (iStent inject, Glaukos Corporation, Laguna Hills, CA), a canalicular scaffold (Hydrus, Ivantis Inc., Irvine, Calif.), or an ab interno suprachoroidal microstent (CyPass, Transcend Medical, Menlo Park, Calif.). Further, the GDDR can be placed onto these devices, or incorporated into their design as a single piece. By so doing, the lumens of the devices can be made larger, with an exponential rise in the potential flow that can be accessed at a later date through laser or mechanical disruption of the flow regulating membrane. Further, multiple devices with the GDDR 200 in place may be placed during one surgical setting, so that some are covered with the GDDR 200 and hence the flow restricted until such time that the flow is needed. Alternatively, multi-lumen shunts can be incorporated into devices which drain into Schlem's canal, the subconjunctival space, and the suprachoroidal space, with the GDDR covering the lumens. As further reduction in intraocular pressure is required, the covered lumens 210 can be accessed with laser to the flow restricting membrane.

Lumen 210 can be further generally configured to maintain aqueous flow with the shunt tube(s) 215. In this regard, the present disclosure can comprise a plurality of lumens 210, or multi-lumen or bifurcated lumens 210. In various embodiments, a plurality of separate lumens 210 is configured to sealingly-engage with a plurality of separate shunt tubes 215.

Moreover, whether in connection with an initial surgery (e.g., as an integrated system) or for use with devices which have been previously implanted, illustrative aqueous shunting systems in accordance with the present disclosure can comprise one or more shunt tubes 215 and/or reservoirs 220 to receive the flow of aqueous. In an example embodiment, shunt tube 215 can have an outer diameter of approximately 0.635 mm (23 g), and an inner diameter of approximately 0.31 mm (30 g). Moreover, any suitable inner/outer diameter shunt tube may be used. Notwithstanding the foregoing, in various embodiments, the present disclosure provides systems comprising one or more shunt tubes 215 having smaller or larger diameters than those taught in the prior art, or multi-lumen or bifurcated shunt tubes 215. By way of non-limiting example, a larger diameter, for example 20 gauge or 18 gauge or greater, shunt tube 215 (or a multi-lumen or bifurcated shunt tube 215) can be configured to allow for greater aqueous flow months or years after surgical implantation (e.g., when the patient's disease worsens) in cases where the high aqueous flow immediately post-operatively would be prohibitive. In this regard, one or more shunt tubes 215 having smaller or larger diameters than those taught in the prior art, or multi-lumen or bifurcated shunt tubes 215 can be implanted, and membrane 205 of GDDR 200 subsequently perforated as needed to increase the flow of aqueous into the one or more shunt tubes 215 and/or reservoirs 220.

Stated another way, in an example embodiment, the inner diameter of shunt tube 215 can be configured to be greater than the maximum diameter that could be used on a patient at the time of operation if the operation was performed without the membrane of the present disclosure. Without membrane 205 of the present disclosure, a shunt tube with too great an inner diameter would allow too much flow. In contrast, with GDDR 200 of the present disclosure, the inner diameter of shunt tube 215 can be greater than the maximum diameter that could be used on a patient at the time of operation because the flow is restricted by membrane 205 in addition to the inner diameter of shunt tube 215. Moreover, the same shunt tube 215 can continue to be used at a subsequent time when additional perforation increases the flow of aqueous. Thus, subsequent adjustments can be made with minimal surgery impact on the patient.

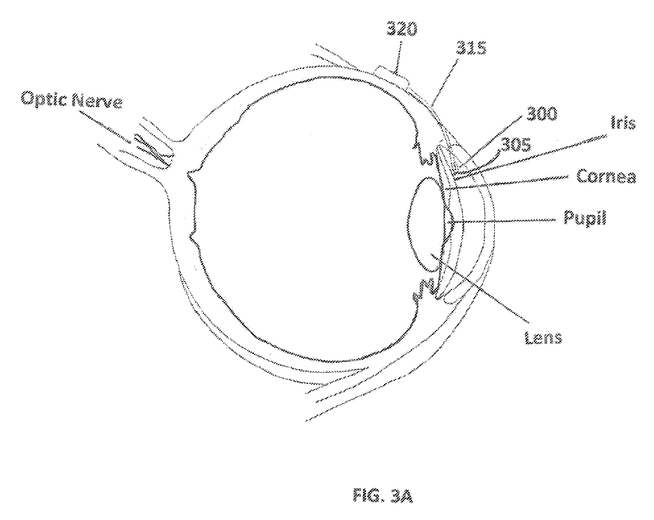

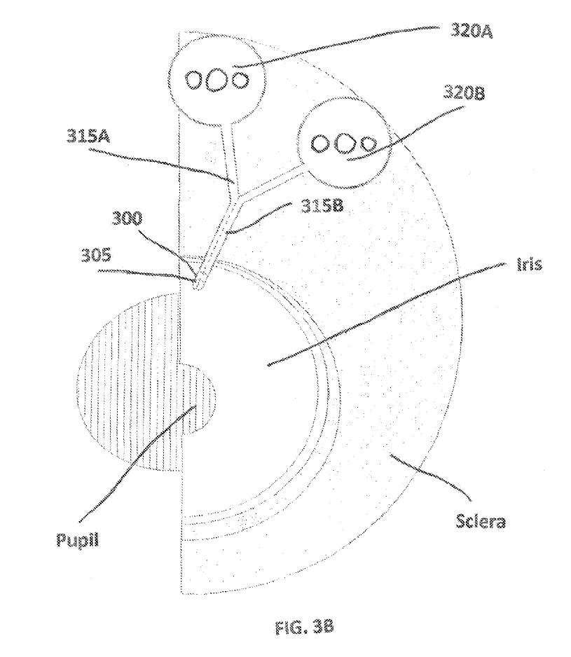

FIG. 3A illustrates an example GDDR 300 in accordance with the present disclosure implanted in connection with aqueous shunting. In an example embodiment, a membrane 305 of GDDR 300 is angled to face the cornea, and thereby allow the surgeon easier surgical access to the face of membrane 305 in order to use a laser or other device to create perforations. GDDR 300 can be like a small cap that can be applied to (or removed from) any existing GDD tube 315 and/or reservoir 320.

In an example embodiment, GDDR 300 may be particularly useful for cases of glaucoma shunt tubes 315 and/or reservoirs 320, including ahmed, malteno, and krupin devices, as well as both fornix and limbus based trabeculectomy procedures.

With reference to FIG. 3B, and as noted above, a multi-lumen or bifurcated shunt tube 315 can be configured to allow for greater aqueous flow months or years after surgical implantation. In example embodiments, membrane 305 of GDDR 300 can comprise a divider (e.g., a divider 106 as shown in FIG. 1), which is configured to allow a medical practitioner to perforate specific areas selectively, and thereby selectively direct the flow of aqueous into one or more of a plurality of reservoirs 320. In other example embodiments, membrane 305 may comprise a continuous face, in which case the medical practitioner can still perforate specific areas selectively as described above to further reduce intraocular pressure, as desired.

By way of further illustration, and with continued reference to FIG. 3B, certain perforations in membrane 305 can open multi-lumen or bifurcated shunt tube 315A to allow the flow of aqueous into reservoir 320A, while other perforations in membrane 305 can open multi-lumen or bifurcated shunt tube 315B to allow the flow of aqueous into reservoir 320B. As above, the plurality of reservoirs 320 can be placed under the conjunctiva.

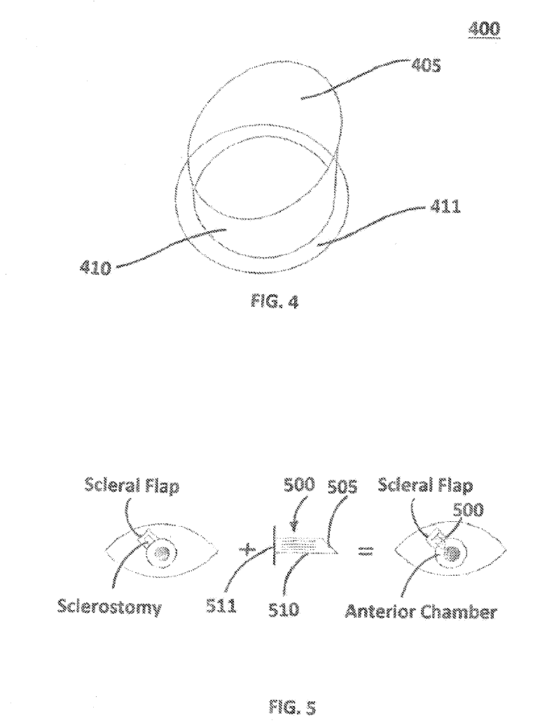

Turning now to FIG. 4, in connection with various embodiments, including those useful with trabeculectomy procedures, a GDDR 400 can further comprise a flange 411, e.g., for seating GDDR 400 at the sclerostomy in trabeculectomy surgery. In an example embodiment, flange 411 comprises a ring shape. In an example embodiment, flange 411 is circumferentially coupled with lumen 410. Flange 411 can be configured to circumferentially secure a lumen 410 and a membrane 405 on one or both opposing sides of one or more sclerostomy openings. In this regard, all or substantially all aqueous flowing through the sclerostomy opening(s) would flow through lumen 410 and membrane 405. More generally, flange 411 can be configured to secure lumen 410 and membrane 405 with respect to one or more sclerostomy openings, or within any other alternate pathway for aqueous flow from an anterior chamber, and thereby direct flow through lumen 410 and membrane 405.

Like lumen 410, flange 411 of GDDR 400 can be comprised of one or more biocompatible materials such as silicone, acrylic, PMMA, fluorinated ethylene propylene, stainless surgical steel, shape memory polymers, collamers, PVDF, bioidentical plant, animal or human cells, and the like. Flange 411 may have holes which allow the passage of sutures or other materials to secure the implant to sclera or other tissue. Alternatively, flange 411 may be secured with a biocombatible adhesive.

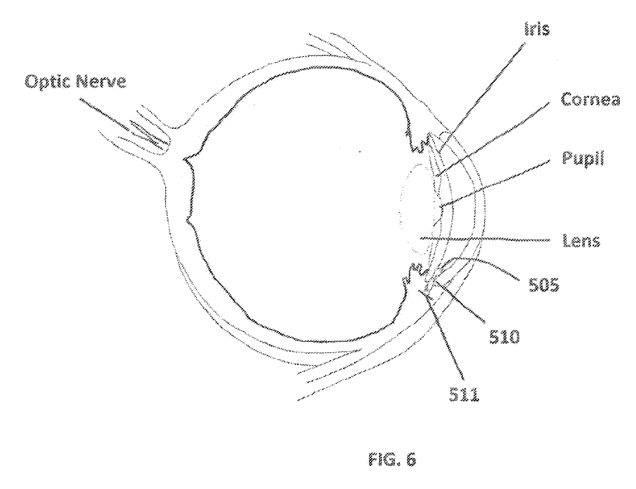

With reference to FIGS. 5 and 6, GDDR 500 comprising a lumen 510 and a flange 511 can be used in connection with trabeculectomy procedures by placing it beneath the scleral flap, through the sclerostomy with its tip into the anterior chamber. In such a configuration, membrane 505 will prevent aqueous flow until such time post-operatively that the medical practitioner determines the conjunctival wounds to be stable. Membrane 505 can then be perforated as clinical need dictates. Current trabeculectomy surgeries typically use a Kelley punch with an opening of 1-3 mm. In various embodiments, the present disclosure provides systems comprising one or more sclerostomy openings having smaller or larger diameters than those taught in the prior art. By way of non-limiting example, a larger diameter, for example 20 gauge or 18 gauge or greater, sclerostomy opening can be configured to allow for greater aqueous flow months or years after surgical implantation (e.g., when the patient's disease worsens) in cases where the high aqueous flow immediately post-operatively would be prohibitive. In this regard, one or more sclerostomy openings having smaller or larger diameters than those taught in the prior art, or multi-lumen or bifurcated sclerostomy openings can be implanted, and membrane 505 of GDDR 500 subsequently perforated as needed to increase the flow of aqueous into the one or more sclerostomy openings.

Stated another way, in an example embodiment, the sclerostomy opening inner diameter is configured to be greater than the maximum diameter that could be used on a patient at the time of operation if the operation was performed without the membrane of the present disclosure. Without the membrane of the present disclosure, a sclerostomy opening with too great an inner diameter would allow too much flow. In contrast, with the GDDR of the present disclosure, the sclerostomy opening inner diameter can be greater than the maximum diameter that could be used on a patient at the time of operation because the flow is restricted by membrane 505 in addition to the inner diameter of the sclerostomy opening. Moreover, the same sclerostomy opening can continue to be used at a subsequent time when additional perforation increases the flow of aqueous. Thus, subsequent adjustments can be made with minimal surgery impact on the patient.

Each of the membrane, lumen(s), shunt tube(s), reservoir(s), and flange can be temporarily or permanently coupled to one or more of the others by adhesion, compression fit, threading, suture, glue, thermal bonding, nitinol, biocompatible adhesive or other shape memory clips, and the like. Likewise, any plurality of the membrane, lumen(s), shunt tube(s), reservoir(s), and flange can be integral one with another. For example, a membrane and a lumen comprise a single piece formed from a single mold, extruded together, etc. In example embodiments, a coupling is configured to maintain coupled elements firmly in place relative to one another even when subjected to shaking and acceleration/deceleration movements.

Illustrative methods for treating a patient having glaucoma, or otherwise lowering intraocular pressure, comprise implanting a GDDR as described supra within an alternate pathway for aqueous flow from an anterior chamber, according to conventional surgical techniques for implanting a GDD, wherein perforations in an implantable membrane of the GDDR increase aqueous flow to lower intraocular pressure within the anterior chamber. Illustrative methods can further comprise evaluating the patient's intraocular pressure at a later time (e.g., hours, days, weeks, months or years later), and further perforating the implantable membrane as needed to further lower the patient's intraocular pressure.

Example embodiments further comprise decreasing the intraocular pressure within the anterior chamber by at least about 1%, more preferably at least about 5%, most preferably at least about 20%. Example embodiments further comprise decreasing the intraocular pressure within the anterior chamber by at least 1 mmHg, 2 mmHg, 4 mmHg or more, to at least about 16 mmHg, more preferably at least about 14 mmHg, most preferably about 10 mmHg, or an otherwise normal or improved intraocular pressure. Example embodiments comprise decreasing the intraocular pressure within the anterior chamber for at least about 2 weeks, or at least about 3-6 months, or at least about 1 year, or at least about 1 decade, or more.

### EXAMPLES

### Example 1

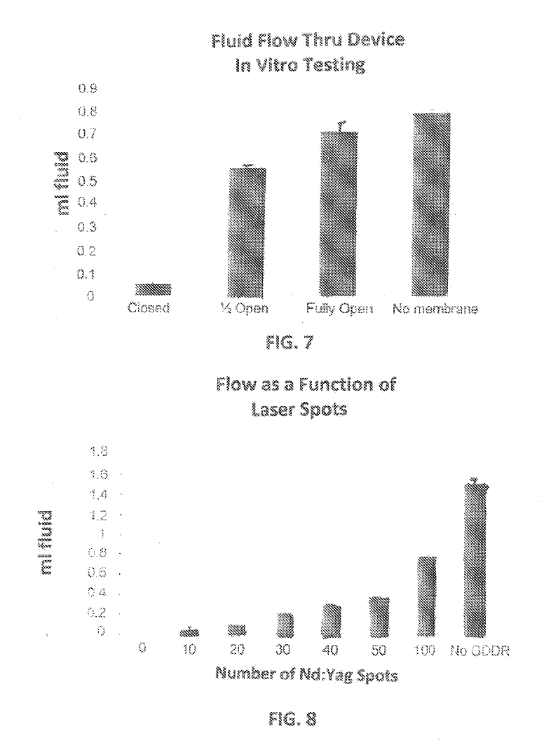

Testing the GDDR in a model eye. The GDDR device was placed over the tip of a conventional GDD, and the tube placed into the model eye through a port. A second port was used to infuse fluid into the eye to maintain a physiologic pressure of 20 mmHg. The amount of fluid which passed through the tube was measured for 30 seconds. The membrane was placed initially with no laser perforations, then with enough laser to open half the membrane, and then more laser to open the membrane completely. Further, the tube was tested with no GDDR in place as a control. Three measurements were done for each configuration, and the results averaged. As shown in FIG. 7, increasing number of laser perforations allows for a titrable amount of flow through the tube of the GDD.

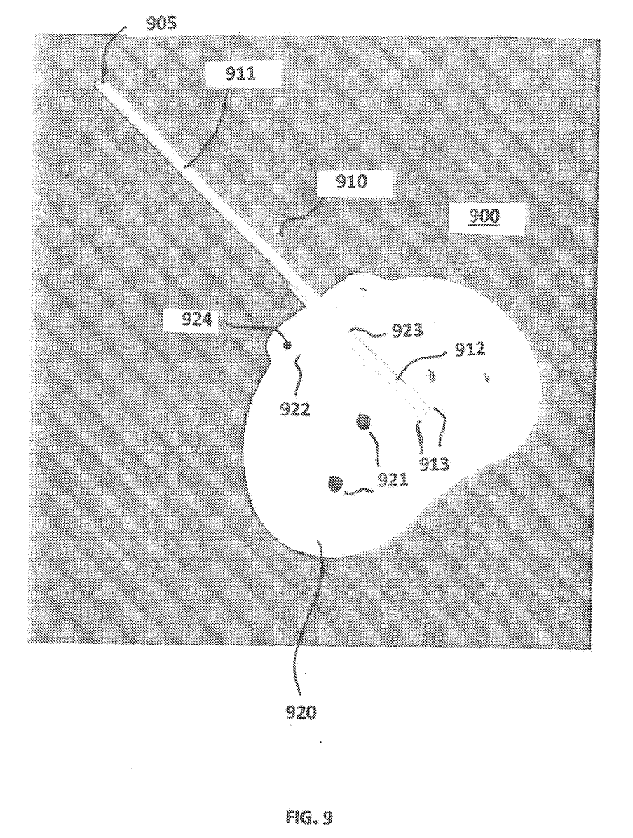

The GDDR was tested ex-vivo in an enucleated porcine eye. The device was placed over the tip of a conventional GDD, and the tube placed into the eye through a corneal paracentisis. An infusion line was used to infuse saline into the eye to maintain a physiologic pressure of 20 mmHg. The amount of fluid which passed through the tube was measured for 60 seconds. The membrane was placed initially with no laser perforations, then with increasing amounts of laser to perforate the membrane, and then more laser to open the membrane completely. Further, the tube was tested with no GDDR in place as a control. Three measurements were done for each configuration, and the results averaged. As shown in FIG. 8, increasing number of laser perforations allows for a titrable amount of flow through the tube of the GDD.

In an example embodiment, a GDDR was configured to be compression fit over the top of a shunt tube. The GDDR was then subjected to stress testing. An example GDDR, composed of a 22 gauge silicone catheter with a 10 nm PVDF membrane, was placed over the tip of a standard 23 gauge silicone drainage tube from a GDD. The GDDR was easily placed on the tip using standard ophthalmic forceps. Once in place, the tube was subjected to shaking and acceleration/deceleration movements in an attempt to dislodge the GDDR. The GDDR remained firmly in place with the force of friction between its inner lumen and the outer lumen of the tube shunt.

As it relates to a further surgical technique using ex-vivo porcine eyes, the GDDR was placed over the tip of a standard tube shunt, which was then inserted into the anterior chamber of a porcine eye through a limbal paracentensis. With the GDDR in place, the tube passed easily through the wound and remained in place in the anterior chamber. Alternatively, the tube without the GDDR was first placed into the anterior chamber, and then the GDDR passed through the same wound in the anterior chamber. Conventional forceps were then used to place the GDDR on the tube of the GDD.

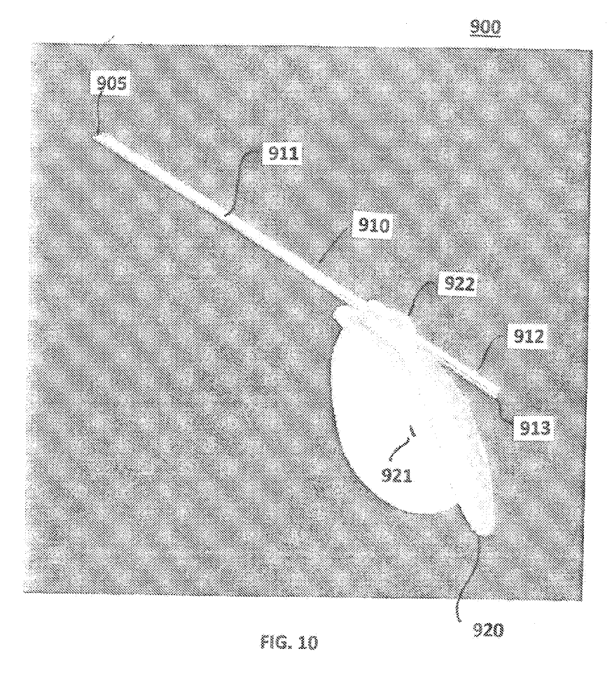

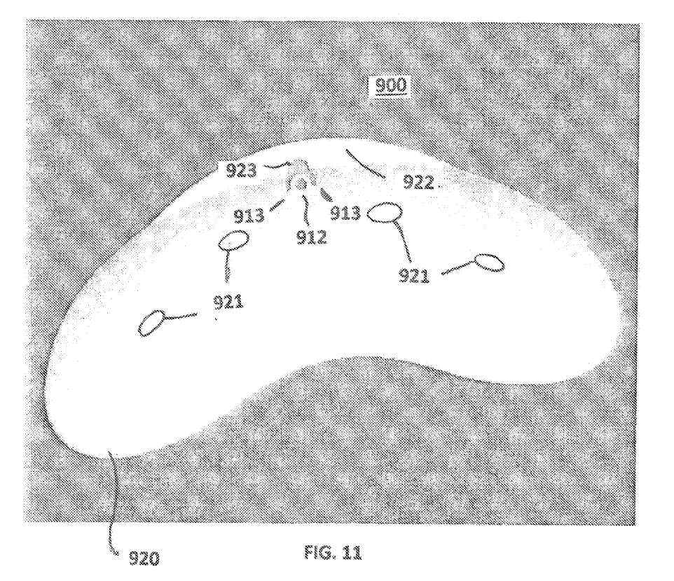

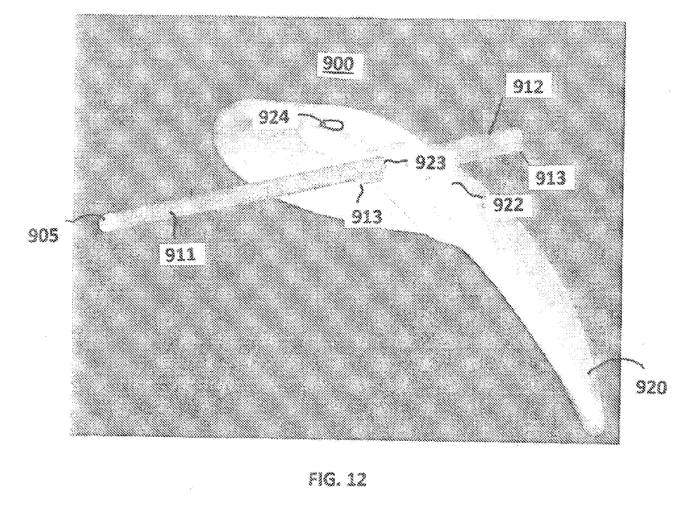

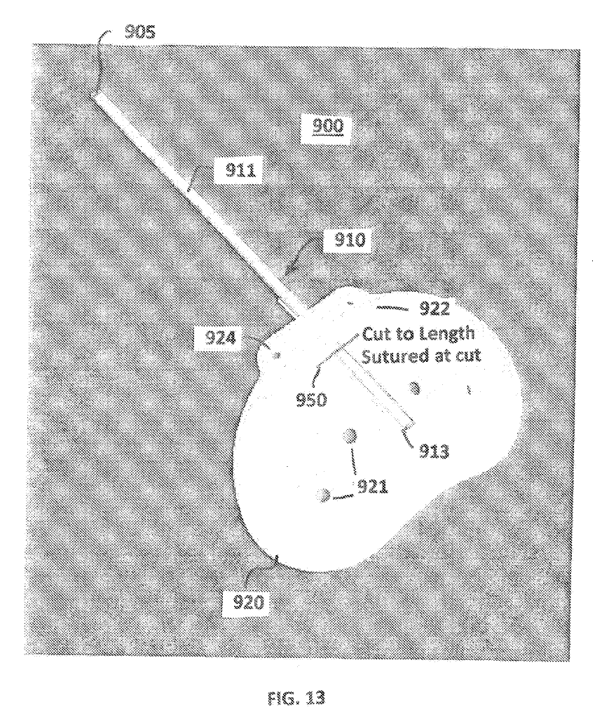

Turning now to FIGS. 9-13, in connection with various embodiments, a shunt tube 910 of a GDDR 900 is illustrated. The GDDR 900 of this embodiment may comprise a reservoir 920 having one or more reservoir holes 921, one or more suture openings 924 a ridge 922 with an aperture 923 configured to mate snuggly with a proximal end 912 of a shunt tube 910. The proximal end 912 of shunt tube 910 may include one or more wing protrusions 913 that run along the proximal end 912 of the shunt tube 910. The one or more wing protrusions 913 configured to mate snuggly with mating aperture 923 in reservoir ridge 922 to prevent twisting movement of shunt tube 910 when snuggly mated with ridge aperture 923. Shunt tube 910 may also comprise a distal end 911 comprising a membrane 905 that is configured such that it allows no aqueous flow prior to perforation, or it may be permeable to low amounts of aqueous flow prior to perforation.

Shunt tube 910 may comprise distal end 911 having membrane 905 and proximal end 912 having one or more wing protrusions 913, wherein shunt tube 910, membrane 905 and one or more wing protrusions 913 are a single integral shunt tube 910. Integral shunt tube 910 may be comprised of one or more biocompatible materials such as PVDF; silicone; filtration and nanofiltration membranes; nucleopore membranes; PMMA; dialysis membranes; cellulose; acrylic; fluorinated ethylene propylene; shape memory polymers; non-reactive polymers; collamers; nylon; bioidentical plant, animal or human living cells, and the like. Accordingly, in example embodiments, integral shunt tube 910 is implantable.

Shunt tube 910 may be made with membrane 905 at the distal end and one or more wing protrusions 913 at the proximal end by as a single piece from a mold, extrusion, etc. Alternatively, shunt tube 910, membrane 905 and one or more wing protrusions 913 may be temporarily or permanently coupled to one or more of the others by adhesion, compression fit, threading, suture, glue, thermal bonding, nitinol or other shape memory clips, biocompatible adhesive, and the like. Alternatively, shunt tube 910, membrane 905, and one or more wing protrusions 913 may be grown of living cells in a mold or on a biocompatible scaffold. Shunt tube 910 may be a 21, 22 or 23 gauge device to permit the flow capacity and determined by the medical practitioner. Wing protrusions 913 may be any shape to provide a means for the medical practioner to suture the shunt tube 910 to the reservoir 920 and/or to mate with the shape of the aperture 923 in ridge 922. Alternatively, the proximal end 912 of shunt tube 910 may be any shape and aperture 923 may be a similar shape, such that when the proximal end 912 of the shunt tube 910 is mated with aperture 923, the shunt tube 910 is secured against twisting or turning within the aperture 923. The ridge 922 and aperture 923 are a securement device or means configured to secure the shunt tube 910 relative to the reservoir 920.

Reservoir 920 may comprise one or more reservoir holes 921 configured to permit aqueous fluid drained via shunt tube 910 to be reabsorbed at a predetermined rate. Reservoir 920 may also comprise one or more suture openings 924 configured to permit the medical practitioner to fix the GDDR 900 into place within the ocular structure during placement to prevent movement within the eye post surgery. Reservoir 920 may comprise a ridge 922 configured with an aperture 923 of a size and shape to snuggly mate with the proximal end 912 and the one or more wing protrusions 913 in such a manner that the shunt tube 910 is prevented from twisting or rotating within the aperture 923. It will be appreciated that protrusions 913 may be any size or shape, so long as they mate with the size and shape of aperture 923 to prevent rotation of the shunt tube within aperture 923. Accordingly, the medical practitioner is able to implant the GDDR 900 during surgery in such a manner to permit easier access to the face of membrane 905 post surgery in order to use a laser or other device to create perforations in membrane 905 and modify or increase aqueous flow.

Reservoir 920 having one or more reservoir holes 921, one or more suture openings 924 and ridge 922 may comprise a single unit manufactured of a soft, biocompatible material such as silicone; acrylic; PNNA; fluorinated ethylene propylene; stainless surgical steel; shape memory polymers; collamers; PVDF; bioidentical living tissue; and the like. The reservoir 920 may comprise a single unit manufactured by compression molding, extrusion, growing biocompatible or bioidentical tissue on a flexible scaffold in a mold, 3D printing with biocompatible or bioidentical material or living tissue, and the like. Alternatively, reservoir 920 and ridge 922 may be separate elements mated by means of biocompatible adhesive, compression, suture, glue, heating, and the like.

It will be appreciated that with the membrane 905 on the distal end 911 of shunt tube 910, the traditional implantation method of implanting the device and trimming the distal end 911 of the shunt tube 910 cannot be used with the present GDDR 900. Accordingly, the closure membrane 905 on the distal end 911 of shunt tube 910 must be maintained during implantation. This is accomplished by threading the proximal end 912 with wing protrusions 913 into the ridge 922 aperture 923. During the implantation procedure, the reservoir plate 920 is affixed to the periphery of the eyeball, anterior to the pupil. In order to provide access to the face of the membrane 905 post surgery within the patient's anterior chamber, the distal end 911 having membrane 905 is pulled forward towards the anterior chamber until the proper length is achieved. The length of the shunt tube 910 may be reduced by grasping the proximal end 913 of the shunt tube 910 behind the ridge 922 of the reservoir 920 and pulling the shunt tube 910 backwards until the desired length is obtained.

Once the proper length is achieved, the shunt tube 910 is cut on the proximal end 913 of the ridge 922 (a typical cut line is shown in FIG. 13), leaving a sufficient portion of the proximal end 913 as to permit the medical practitioner to place one or more sutures through the proximal end 913 of the shunt tube 910 and the reservoir 920 to secure the shunt tube 910 to the reservoir 920.

The method of securing the shunt tube 910 relative to the reservoir 920 may be accomplished by means of one or more sutures, glue, biocompatible adhesive, heating the ridge 922 to deform it or melt it onto the shunt tube 910, forming the aperture 923 and the shunt tube 910 such that there are mechanical interference or friction components that grip the shunt tube 910 within the aperture 923 against movement under normal conditions. Alternatively, an oversized plug with lumen (not shown) may be inserted into the shunt tube 910 causing the shunt tube 910 and ridge aperture 923 to expand to accommodate the plug, creating a snug fit between the shunt tube 910 and the aperture 923. Alternatively, if tissue or tissue over a scaffold is utilized for the shunt tube 910, the reservoir 920, or both, the tissues employed may be selected or engineered such that they adhere or grow together within a short time of implantation.

Further, the reservoir plate can be augmented. The main plate is attached to the previously mentioned securement device that allows the tube to be adjusted in length. The main reservoir plate is equipped with attachment areas so that sub-plates may be attached to any or all of the three sides away from the tube attachment area. This allows custom fitting and sizing of the reservoir plate to allow the surgeon to adjust the implant to various globe sizes, anatomic configuration, previous surgeries, and even to different species such as needed in veterinary ophthalmic procedures for dogs, cats, and the like.

As the present GDDR is intended to improve control over increases in interocular pressure without requiring frequent replacement of the device or repetitive surgeries, designs may be implemented to permit greater flow beyond the 22 gauge design. This increased flow design may include one or more shunt tubes 911 to one or more reservoirs 920; a double 23 gauge or double 22 gauge shunt tube 910 with matching reservoirs, and the like. A double shunt tube 910 may be coupled to a reservoir 920 with a profile similar to the symbol for infinity.

The various embodiments may be utilized on human patients, as well as other animals known to develop intraocular pressure. It will be appreciated that the components may necessitate sizing to accommodate larger or smaller patients, the fundamental principles and teachings are taught for human and non-human animals requiring relief from excessive intraocular pressure.

### Example 2

In vivo testing of a large lumen glaucoma drainage device. A large lumen glaucoma drainage device (LL-GDD) equipped with a flow regulator was prepared and tested in vivo. The device's membrane can be non-invasively opened with laser in the post-operative period to adjust aqueous flow and intraocular pressure, as clinical conditions demand.

In Vitro Testing:

The LL-GDD was tested first in a model eye equipped with ports for infusion and pressure measurement. With the membrane face intact, there was an average of 25.5±0.3 μL balanced salt solution (BSS) drained, with a mean flow rate of 0.9 μL/sec. With the membrane face completely open, the total BSS drained averaged 4023.3 μL+/−38.4 μL and a flow rate of 134.1 μL/sec. In vivo testing: New Zealand white satin cross rabbits were used, two eyes receiving the LL-GDD and the two fellow eyes serving as the control group with no intervention performed. After the procedure, the TOP in the LL-GGD surgical group dropped an average of 5.5 mmHg (p=0.001) which was maintained until the membrane laser procedure at week five resulting in an average TOP reduction of 1.8 mmHg. At week seven, the average IOP in the surgical group was 11 mmHg compared to 18 mmHg in the control group (p<0.001). A second laser procedure was done to completely open the membrane face, which resulted in an immediate drop in the average IOP of the surgical group by another 2.7 mmHg, which was maintained until the study termination at day 55.

As noted above, trabeculectomy is the most frequently performed filtering operation and remains one of the most effective, but it can be complicated by choroidal detachment or endophthalmitis, even years after surgery. Glaucoma drainage devices (GDD) have shown an advantage in maintaining IOP control compared to trabeculectomy for patients with uncontrolled IOP after previous incisional surgeries. This has resulted in an increased interest in the use of GDD for the management of glaucoma and is the option of choice for many types of glaucoma such as neovascular, uveitic, iridocorneal endothelial syndrome, glaucoma related to penetrating keratoplasty, keratoprosthesis or following retinal detachment repair.

The most common early complications of tube shunt implantation are hypotony and associated problems. The Glaucoma Drainage Device Regulator (GDDR) implant used in these studies was designed to overcome these hurdles. It allows the surgeon to control the rate of flow through the device non-invasively in the post-operative period, allowing customized treatment for patients.

Current commercially-available shunts typically use a silicone tube with an outer diameter of 0.64 mm (23 GA) and an inner diameter of 0.34 mm (30 GA). We are describing and testing a second generation device with an increased lumen size: the large lumen glaucoma drainage device (LL-GDD) which has an outer diameter of 0.72 mm (22 GA) and an internal diameter of 0.5 mm. This represents an increase in the outer diameter of 13% (0.08 mm) and an increase in the inner diameter of 47% (0.16 mm)—which translates into a quadrupling of flow as described by Poiseuille's law whereby there is an exponential increase in flow with relation to the tube radius.

With conventional implant hardware designs, this enlarged lumen device could not be safely placed in an eye since the high rate of uncontrolled flow in the immediate post-operative period would lead to profound hypotony. But using the glaucoma drainage device regulator (GDDR) technology, this additional flow can be controlled and held in reserve. That is, post-operatively the flow is restricted by the device's membrane which covers the lumen of the drainage device. As clinical conditions demand, the membrane can be non-invasively opened with laser. The membrane reduces, but does not totally restrict flow when completely intact. This is advantageous as it allows immediate TOP control, as well as keeping aqueous flowing through the device to prevent blockage or failure of the GDD and to prevent infection.

In vivo testing: In vivo tests were conducted to demonstrate: successful surgical implantation, prevention of immediate post-operative hypotony, increased flow on demand post-implantation, and to compare flow rates to conventional drainage devices.

Large lumen glaucoma drainage devices (LL-GDD) of this disclosure were constructed using 22 g silicone angiocatheters. A 10 nm PVDF membrane was then affixed to the end using cyanoacrylate. PVDF was chosen given its long track record of biocompatibility and previous use in intraocular lens designs. Further, the membrane's thickness allows it to be easily ruptured using either thermal or photodisruptive lasers. Using a standard Baerveldt (Abbott Laboratories, Abbott, Ill.) drainage device, the standard 23 g tube was removed and the 22 g tube affixed to the reservoir plate.

The (LL-GDD) was tested first in a model eye equipped with ports for infusion and pressure measurement. Balanced saline solution was hung at the appropriate height to maintain a constant pressure of 25 mmHg, which was monitored during the testing using an industrial grade differential pressure manometer (HD750, Extech Insturments, Nashua, N.H.). The LL-GDD was placed into the system and the amount of fluid which passed through the tube was measured for 30 seconds. The membrane was placed initially with no laser perforations, then with enough laser to progressively open ⅙ of the membrane until 100% of the membrane was opened. An Nd:YAG laser (YC-1600, Nidek, INc, Fremont, Calif.) was used to rupture the PVDF membrane with the following parameters: 4.3 mJ, single pulse. Further, a conventional 23-gauge tube was tested with no regulator in place as a control. Three measurements were done for each configuration, and the results averaged.

New Zealand white satin cross rabbits were used, two eyes receiving the LL-GDD and the two fellow eyes serving as the control group with no intervention performed. For all surgical cases, the conjunctiva was opened at the limbus for three clock hours superonasally and the underlying sclera exposed. To accommodate the decreased size of the rabbit's globe, all of the reservoir plates were cut down 2 mm on each side using a template to ensure consistency. The reservoir plate was affixed to the globe using 8-0 nylon suture. A 22-gauge needle was used to create a tunnel through the sclera and enter the anterior chamber just anterior to the iris. This tunnel was widened slightly in the large lumen device group to accommodate the larger tube. The tubes were then placed in the anterior chamber and the conjunctiva repositioned with vicryl suture. At post-operative weeks five and seven the membrane on the 22 g device was ruptured with argon laser.

In all animals, the right eye underwent surgery and the left eye served as control. All eyes undergoing surgery received topical antibiotic drops for 7 days and topical steroid drops for 2 weeks. Baseline intraocular pressure and anterior segment photos were taken of all eyes, and TOP taken immediately before and after every procedure, as well as twice a week for the eight weeks of the study. A hand-held veterinary model tonometer (Tono-Pen Vet, Reichert Technologies, Depew, N.Y.) was used for this purpose. The drainage devices were left in place for the duration and the animals examined daily for the first week and then weekly thereafter. The student's t-test was used to compare the TOP between groups.

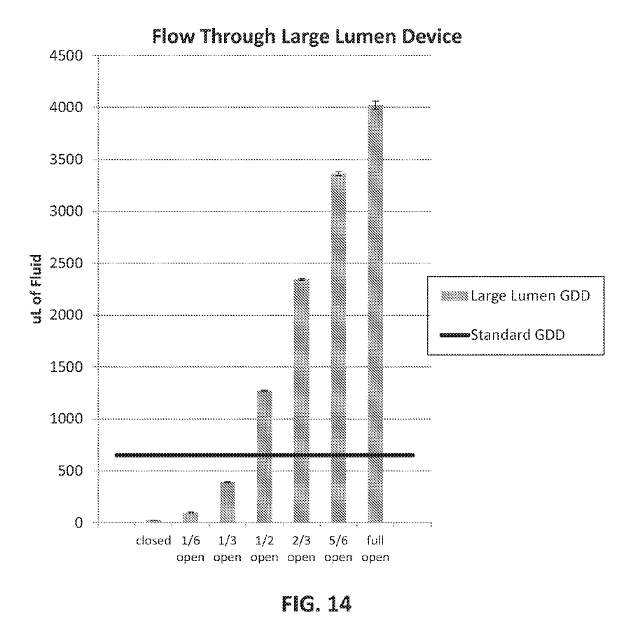

The results of the in vitro test are plotted in FIG. 14. With the membrane face intact, there was an average of 25.5±0.3 μL BSS drained, with a mean flow rate of 0.9 μL/sec. As the membrane face was progressively opened with laser, the flow correspondingly increased in accordance with Poiseuille's law. With the membrane face completely open, the total BSS drained averaged 4023.3 μL+/−38.4 μL and a flow rate of 134.1 μL/sec. Moving from the closed position to the fully open position, there is a three orders of magnitude difference in the potential flow through the LL-GDD. While this is flow rate much higher than would be needed clinically, it demonstrates the ability of the device to overcome resistance around the reservoir plate which may develop years after implantation.

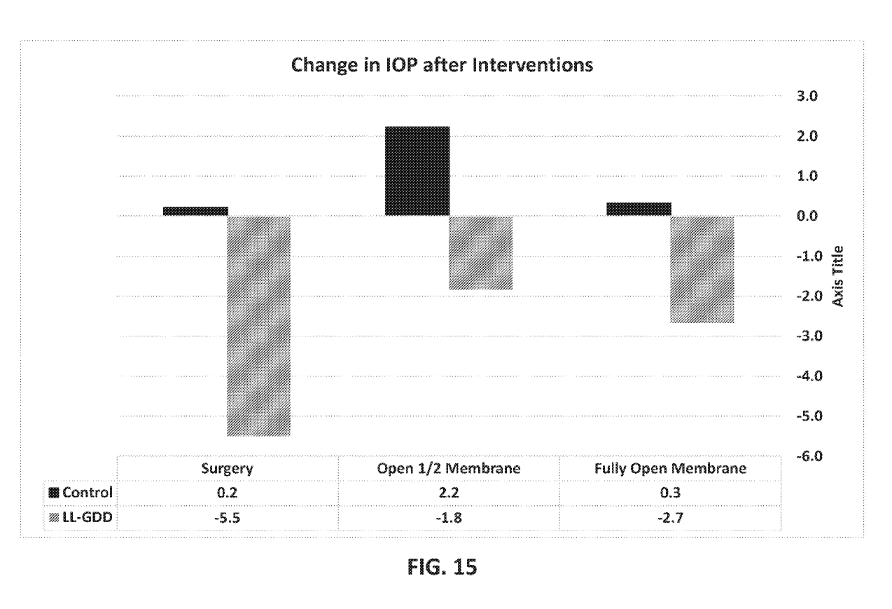

During the 55 days following surgery, none of the study or control eyes showed signs of inflammation, infection or cataract formation on ophthalmologic examination. At baseline, there was no difference in TOP between the control and surgical group (16.8 v. 16.7 mmHg, p=0.49). Immediately after the surgery, the TOP in the LL-GGD surgical group dropped an average of 5.5 mmHg (FIG. 15), a statistically significant reduction (p=0.001) that was maintained until the membrane laser procedure at week five. Despite having a tube with over four times the flow capacity of a conventional glaucoma drainage device, the IOP never dropped precipitously, and no choroidal effusions occurred. It is important to note that the membrane regulator face was completely intact during the first five weeks, indicating the passive flow across the intact membrane was sufficient to have a significant effect on TOP.

At week five, half of the membrane face was ruptured using argon laser. This resulted in an immediate increase in flow as evidenced by a fluid bleb over the reservoir plate, and a reduction in the TOP by an average of 1.8 mmHg in the surgical group (FIG. 15). The two weeks following the initial 50% membrane opening, the average TOP in the control group ranged from 4 to 9 mmHg lower than the control group.

At week seven, the average TOP in the surgical group was 11 mmHg compared to 18 mmHg in the control group (p<0.001). A second laser procedure was done to completely open the membrane face, which resulted in an immediate drop in the average TOP of the surgical group by another 2.7 mmHg (FIG. 15), which was maintained until the study termination at day 55. During the eight weeks following surgery, none of the surgical or control eyes showed signs of inflammation, infection or cataract formation on ophthalmologic examination.

Glaucoma drainage devices provide surgeons a means to lower TOP in patients with medically uncontrolled glaucoma, but their high rate of failure limits their long-term utility. These in vivo studies evaluated a next-generation glaucoma drainage device with quadruple the flow capacity of standard GDDs, as well as the ability to adjust both the post-operative flow as well as the placement of the tube tip in the anterior chamber. The large lumen drainage device disclosed herein is designed to address the two major factors limiting the clinical utility of current GDDs: 1) preventing post-operative hypotony, 2) extending the device's functional duration. The first goal is accomplished with the flow restrictor membranes over the lumen of the LL-GDD. This restricts aqueous flow through the tube until the surgeon has determined that the eye is stable, and the membrane can then be opened non-invasively with laser or mechanically with a needle. The second goal is achieved by having a large lumen device, in effect quadrupling the overall efficacy and potential drainage capability of the device. Whether five months or five years after the initial surgery, this additional flow can be tapped into as a means to further reduce the patient's TOP as dictated by clinical need.

As described above, the membranes regulate flow when completely intact, but do not completely block it—which is a distinct design advantage. This means that there will be a continual, albeit low, flow of aqueous through the second unopened LL-GDD. This prevents blockage or failure of the tube, as well as minimizing the chance of infection.

In terms of controlling TOP, these LL-GDD have several distinct advantages: first, the membrane regulator prevents overfiltration and hypotony in the early post-operative period; and second, additional flow can be tapped into by physically opening the membrane face—we have demonstrated that this can be done either mechanically with a needle, or non-invasively with laser.

In summary, this large-lumen glaucoma drainage device testing clearly demonstrated an ability both to prevent immediate post-operative hypotony and to allow progressively lower TOP. Eight weeks after the initial surgery, the animals exhibited no adverse effects and the surgical group maintained a statistically significant lowering of IOP. Additional studies are underway to further characterize the surgical utility and biocompatibility of this next generation aqueous flow device in the management of glaucoma.

It will be apparent to those skilled in the art that various modifications and variations can be made in the present disclosure without departing from the spirit or scope of the disclosure. For example, while the moniker “glaucoma drainage device regulator” has been used in describing illustrative embodiments, the present disclosure is generally applicable to any treatment aimed at lowering intraocular pressure. Moreover, while example embodiments herein may have been described with reference to only one or the other of aqueous shunting and trabeculectomy procedures, such embodiments can be applied to the other, as well as to unnamed and yet undiscovered procedures. Thus, it is intended that the present disclosure cover the modifications and variations of this disclosure provided they come within the scope of the appended claims and their equivalents.

Likewise, numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of structure, materials, elements, components, shape, size and arrangement of parts including combinations within the principles of the disclosure, to the full extent indicated by the broad, general meaning of the terms in which the appended claims are expressed. To the extent that these various modifications do not depart from the spirit and scope of the appended claims, they are intended to be encompassed therein.

What is claimed is:

1. An implantable glaucoma drainage device regulator, comprising
a shunt tube with an opening at a proximal end and a membrane at a distal end, wherein perforations in the membrane increase aqueous flow to lower intraocular pressure, and wherein the membrane is configured to be selectively perforated; and
a reservoir configured to mate with the shunt tube, wherein the shunt tube and reservoir are configured to be mated in order to enable the membrane to permit shunting of aqueous from the eye to the reservoir.

2. The glaucoma drainage device regulator of claim 1, wherein the reservoir, shunt tube and membrane are configured to enable the membrane to be selectively perforated after implantation.

3. The glaucoma drainage device regulator of claim 2, wherein the reservoir comprises a ridge having an aperture configured to mate with the proximal end of the shunt tube.

4. The glaucoma drainage device regulator of claim 3, wherein the proximal end of the shunt tube comprises one or more protrusions and the aperture in the ridge of the reservoir is configured to mate with the proximal end of the shunt tube with one or more protrusions in a manner to restrain the shunt tube against turning within the aperture.

5. The glaucoma drainage device regulator of claim 4, wherein the reservoir has one or more suture openings configured to permit the reservoir to be sutured to a patient's eye during implantation.

6. The glaucoma drainage device regulator of claim 4, wherein the shunt tube, the membrane and one or more protrusions on the proximal end of the shunt tube are integral and comprised of one or more of PVDF, silicone, filtration and nanofiltration membranes, nucleopore membranes, PMMA, dialysis membranes, cellulose, acrylic, fluorinated ethylene propylene, shape memory polymers, non-reactive polymers, collamers, living tissue, biocompatible material, biocompatible tissue, and nylon.

7. The glaucoma drainage device regulator of claim 4, wherein a surface of the membrane is color coded, numbered, or has writing or another target to indicate one or more areas to perforate.

8. The glaucoma drainage device regulator of claim 4, wherein the implantable membrane is configured to be selectively perforated by photodisruptive or ablative laser.

9. The glaucoma drainage device regulator system of claim 4, wherein the reservoir is comprised of one or more of silicone, acrylic, PMMA, fluorinated ethylene propylene, stainless surgical steel, shape memory polymers, collamers, living tissue, biocompatible material, biocompatible tissue, and PVDF.

10. The glaucoma drainage device regulator system of claim 1, wherein the membrane is comprised of one or more of PVDF, silicone, filtration and nanofiltration membranes, nucleopore membranes, PMMA, dialysis membranes, cellulose, acrylic, fluorinated ethylene propylene, shape memory polymers, non-reactive polymers, collamers and nylon.

11. The glaucoma drainage device regulator system of claim 10, wherein a surface of the membrane is color coded, numbered, or has writing or another target to indicate one or more areas to perforate.

12. A method for lowering intraocular pressure, comprising implanting a membrane within an alternate pathway for aqueous flow from an anterior chamber, wherein the membrane is integral with a shunt tube, wherein perforations in the implantable membrane increase aqueous flow to lower intraocular pressure within the anterior chamber, and wherein the implantable membrane is configured to be selectively perforated by photodisruptive or ablative laser.

13. The method of claim 12, wherein the integral membrane and shunt tube is configured to couple with a reservoir, and wherein the method is used in connection with an aqueous shunting procedure.

14. The method of claim 13, wherein the shunt tube is comprised of one or more of silicone, acrylic, PMMA, fluorinated ethylene propylene, stainless surgical steel, shape memory polymers, collamers, living cells, biocompatible material, biocompatible cells, and PVDF.

15. The method of claim 13, wherein the membrane is at a distal end of the shunt tube and one or more protrusions run along the shunt tube at the proximal end of the shunt tube, wherein the reservoir comprises a ridge having an aperture with a shape to mate with the proximal end of the shunt tube with the one or more protrusions, and wherein the proximal end of the shunt tube is mated to the aperture in the ridge of the reservoir.

16. The method of claim 15, wherein the reservoir comprises suture openings, the method further comprising suturing the reservoir to the eye during implantation in order to secure the reservoir against movement.

17. The method of claim 12, wherein the membrane is comprised of one or more of PVDF, silicone, filtration and nanofiltration membranes, nucleopore membranes, PMMA, dialysis membranes, cellulose, acrylic, fluorinated ethylene propylene, shape memory polymers, non-reactive polymers, collamers, living cells, biocompatible material, biocompatible cells, and nylon.

18. The method of claim 17, wherein a surface of the membrane is color coded, numbered, or has writing or another target to indicate one or more areas to perforate.

19. The method of claim 16, further comprising securing the shunt tube to the reservoir.

20. An implantable glaucoma drainage device regulator system comprising:
a shunt tube having an integral closed, angled membrane at a first end and an open second end;
a reservoir having one or more means for attachment to a surface of an eyeball;
wherein the shunt tube and the reservoir comprise flexible, biocompatible materials;
wherein the reservoir comprises a means for mating with the open second end of the shunt tube and securing the shunt tube against twisting movement; and wherein after the implantable glaucoma drainage device regulator system is implanted, the angled membrane is accessible for perforation without surgery.
